(19) United States
(12) Patent Application Publication
(10) Pub. No.: US 2024/0412825 A1
(43) Pub. Date: Dec. 12, 2024

(54) METHOD AND SYSTEM FOR DETERMINING BIOCHAR IDENTITY AND ELECTRONIC DEVICE

(71) Applicant: Zhejiang University of Science & Technology, Hangzhou City (CN)

(72) Inventors: Zhong CHENG, Hangzhou City (CN); Shengdao SHAN, Hangzhou City (CN); Xikun GAI, Hangzhou City (CN); Yefeng ZHANG, Hangzhou City (CN)

(21) Appl. No.: 18/501,461

(22) Filed: Nov. 3, 2023

(30) Foreign Application Priority Data
Jun. 12, 2023 (CN) 2023106920302

(51) Int. Cl. G16C 20/20; G16C 20/70

(52) U.S. Cl. CPC G16C 20/20; G16C 20/70

(57) ABSTRACT

The present disclosure discloses a method and system for determining biochar identity and an electronic device. The method includes: inputting physical and chemical property data of a sample to be identified into a biochar identity determination model to obtain identity information of the sample, where a process for determining the biochar identity determination model includes the following steps: performing abnormality detection and standardization processing on the input variable data matrix, and constructing a feature data matrix based on a processed input variable data matrix; and obtaining a random subspace nearest neighbor clustering ensemble learning classifier based on the feature data matrix, a sample identity multi-category label column vector and a random subspace nearest neighbor clustering ensemble learning algorithm, where the random subspace nearest neighbor clustering ensemble learning classifier is the biochar identity determination model.

### CROSS-REFERENCE TO RELATED APPLICATION

This patent application claims the benefit and priority of Chinese Patent Application No. 2023106920302, filed with the China National Intellectual Property Administration on Jun. 12, 2023, the disclosure of which is incorporated by reference herein in its entirety as part of the present application.

### TECHNICAL FIELD

The present disclosure relates to the technical field of biochar detection and pattern recognition, and in particular, to a method and system for determining biochar identity and an electronic device.

### BACKGROUND

Biochar is a carbon-rich porous solid material obtained by pyrolysis or oxidation of a biomass raw materials in an anaerobic or low oxygen environment at a high temperature (usually between 300° C. and 700° C.), which has good antioxidation, heat resistance and adsorption capacity.

Transforming waste biomass (such as crop straw, livestock manure, forestry wastes, perishable garbage, industrial sludge, and other wastes) into biochar through pyrolysis not only can effectively reduce the pollution and greenhouse gases emissions caused by waste biomass, but also serve as a soil amendment to increase the content of organic matter in soil, improve the soil structure, promote plant growth, and realize green and sustainable development of environmental protection and resource recycling. In addition, the production of biochar provides employment opportunities for rural areas and promotes rural economic development and common prosperity.

Due to differences in raw material types, technical methods, pyrolysis processes, and the like, biochar shows great differences in physical and chemical properties such as structure, composition, pore volume, and specific surface area, which in turn makes the biochar have different environmental effects. While the application field of biochar is expanding, biochar with different properties such as carbon storage value, fertility value, lime equivalent value and particle size is classified to standardize product quality of the biochar, facilitate the selection of suitable biochar for application, promote the diversification, standardization and serial production of biochar products, and assist in sustainable development of biochar industrialization. However, at present, there is no efficient and accurate method for determining biochar identity.

### SUMMARY OF THE INVENTION

An objective of the present disclosure is to provide a method and system for determining biochar identity and an electronic device, which can efficiently and accurately identify information of the biochar.

To achieve the above objective, the present disclosure provides the following technical solutions:

In a first aspect, the present disclosure provides a method for determining biochar identity, including:
obtaining physical and chemical property data of a sample to be identified, where the samples include waste biomass and biochar corresponding to the waste biomass, the biochar is a solid material obtained by carbonizing the waste biomass, and the physical and chemical property data includes a hydrogen content, an organic carbon concentration, a nitrogen content, a phosphorus content, a potassium content, a degree of acidity or alkalinity (pH scale), a specific surface area, and a pore volume; and
inputting the physical and chemical property data of the sample into a biochar identity determining model to obtain identity information of the sample where
a process for determining the identity determination model of biochar includes the following steps:
constructing high-dimensional multi-category biochar sample data, where the high-dimensional multi-category biochar sample data includes an input variable data matrix and an identity multi-category label column vector;
performing abnormality detection and standardization processing on the input variable data matrix in the high-dimensional multi-category biochar sample data to obtain a processed input variable data matrix;
constructing a feature data matrix based on the processed input variable data matrix; and
obtaining a random subspace nearest neighbor clustering ensemble learning classifier based on the feature data matrix, the sample identity multi-category label column vector and a random subspace nearest neighbor clustering ensemble learning algorithm, where the random subspace nearest neighbor clustering ensemble learning classifier is the biochar identity determination model.

In a second aspect, the present disclosure provides a system for determining biochar identity, including:
a sample data acquisition module, configured to obtain physical and chemical property data of a sample to be identified, where the sample includes waste biomass and biochar corresponding to the waste biomass, the biochar is a solid material obtained by carbonizing the waste biomass, and the physical and chemical property data includes a hydrogen content, an organic carbon concentration, a nitrogen content, a phosphorus content, a potassium content, a pH scale, a specific surface area, and a pore volume; and
a biochar identity information determining module, configured to input the physical and chemical property data of the sample into the biochar identity determination model to obtain identity information of the sample, where
a process for determining the identity determination model of biochar includes the following steps:
constructing high-dimensional multi-category biochar sample data, where the high-dimensional multi-category biochar sample data includes an input variable data matrix and an identity multi-category label column vector;
performing abnormality detection and standardization processing on the input variable data matrix in the high-dimensional multi-category biochar sample data to obtain a processed input variable data matrix;
constructing a feature data matrix based on the processed input variable data matrix; and
obtaining a random subspace nearest neighbor clustering ensemble learning classifier based on the feature data matrix, the sample identity multi-category label column vector and a random subspace nearest neighbor clustering ensemble learning algorithm, where the random subspace nearest neighbor clustering ensemble learning classifier is the biochar identity determination model.

In a third aspect, the present disclosure provides an electronic device, including a memory and a processor, where the memory is configured to store a computer program, and the processor runs the computer program to enable the electronic device to perform the method for determining biochar identity according to the first aspect.

According to specific embodiments of the present disclosure, the present disclosure has the following technical effects:

According to the present disclosure, main physical and chemical property data of waste biomass and carbonized biochar thereof from different regions, of different raw materials and at different temperatures are first experimentally sampled, analyzed and measured, and then a biochar identity determination model is established by using a random subspace nearest neighbor clustering ensemble learning method. The method has the advantages of high accuracy, good result stability, strong generalization ability, good extensibility, and the like.

### BRIEF DESCRIPTION OF THE DRAWINGS

To describe the technical solutions in embodiments of the present disclosure or in the prior art more clearly, the accompanying drawings required in the embodiments are briefly described below. Apparently, the accompanying drawings in the following description show merely some embodiments of the present disclosure, and other drawings can be derived from these accompanying drawings by those of ordinary skill in the art without creative efforts.

FIG. 1 is a flowchart of a method for determining biochar identity according to an embodiment of the present disclosure;

FIG. 2 is a schematic structural and functional diagram of a random subspace nearest neighbor clustering ensemble learning classifier according to an embodiment of the present disclosure;

FIG. 3 is a flowchart of the implementation of a random subspace nearest neighbor clustering ensemble learning classifier for biochar identity determination according to an embodiment of the present disclosure;

FIG. 4 is an importance ranking diagram of input variables according to an embodiment of the present disclosure; and

FIG. 5 is a diagram showing a result of biochar identity determination by using sample data by a random subspace nearest neighbor clustering ensemble learning classifier according to an embodiment of the present disclosure.

### DETAILED DESCRIPTION OF THE EMBODIMENTS

The technical solutions of the embodiments of the present disclosure are clearly and completely described below with reference to the accompanying drawings in the embodiments of the present disclosure. Apparently, the described embodiments are merely some rather than all of the embodiments of the present disclosure. All other embodiments obtained by those skilled in the art based on the embodiments of the present disclosure without creative efforts shall fall within the protection scope of the present disclosure.

In order to make the above objective, features and advantages of the present disclosure clearer and more comprehensible, the present disclosure will be further described in detail below in combination with accompanying drawings and specific implementations.

### Embodiment 1

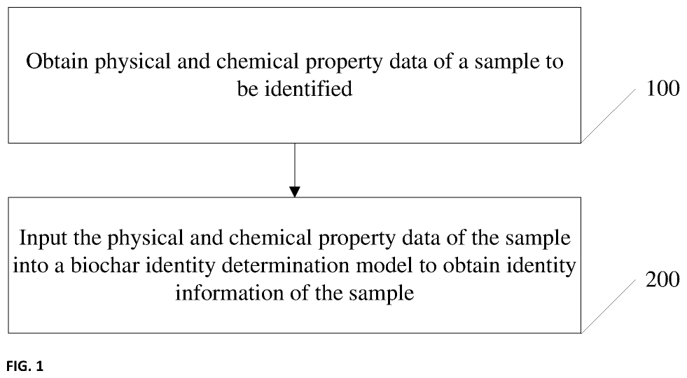

As shown in FIG. 1, a method for determining biochar identity according to this embodiment includes the following steps.

Step 100: Obtain physical and chemical property data of a sample to be identified, where the sample includes waste biomass and biochar corresponding to the waste biomass. The biochar is a solid material obtained by carbonizing the waste biomass, and the physical and chemical property data includes a hydrogen content, an organic carbon concentration, a nitrogen content, a phosphorus content, a potassium content, a pH scale, a specific surface area, and a pore volume.

Step 200: Input the physical and chemical property data of the sample into a biochar identity determination model to obtain identity information of the sample.

A process for determining the identity determination model of biochar includes the following steps.

Step S1: Construct high-dimensional multi-category biochar sample data.

Step S2: Perform abnormality detection and standardization processing on the input variable data matrix in the high-dimensional multi-category biochar sample data.

Step S3: Construct a feature data matrix based on the processed input variable data matrix.

Step S4: Obtain a random subspace nearest neighbor clustering ensemble learning classifier based on the feature data matrix, the sample identity multi-category label column vector and a random subspace nearest neighbor clustering ensemble learning algorithm, where the random subspace nearest neighbor clustering ensemble learning classifier is the biochar identity determination model.

In this embodiment, in step S1, a plurality of pieces of important physical and chemical property data collected from experiments of sample waste biomass and the carbonized sample biochar thereof are sorted out, and thus high-dimensional input vectors of the sample data and multi-category attribute labels of identity of the sample waste biomass and the carbonized sample biochar thereof are established, including the following specific processes.

Step S11: Screen physical and chemical property parameters of the sample to be identified, where the sample includes waste biomass and biochar obtained by carbonizing the waste biomass. In this step, different types of physical and chemical property parameters such as a carbon storage value, a fertility value, a potential of hydrogen and particle size distribution are selected based on application values of the waste biomass raw material and the carbonized biochar thereof.

Step S12: Define specific physical and chemical property parameters to obtain a plurality of physical and chemical property indexes, where the carbon storage value includes physical and chemical property indexes such as a hydrogen (H) content (%) and an organic carbon concentration Corg (%); the fertility value includes physical and chemical property indexes such as a nitrogen (N) content, a phosphorus (P) content, and a potassium (K) content (%); the potential of hydrogen includes a physical and chemical property index which is a pH scale; and the particle size distribution includes physical and chemical property indexes such as a specific surface area BET (m²/g) and a pore volume Vpore (cm³/g).

Step S13: Arrange the high-dimensional input vectors, that is, sequentially denote the plurality of physical and chemical property indexes defined in step S12 as x₁, x₂, . . . , xₚ (p is a number of physical and chemical property indexes), and assemble in the form of row vectors to form input vectors x=[x₁, x₂, . . . , xₚ] of the individuals.

Step S14: Experimentally collect the input variable data matrix, that is, select sample waste biomass from different regions and of different raw materials and sample biochar respectively prepared at different carbonization temperatures, experimentally measure p pieces of physical and chemical property data defined in step S12, arrange an input vector x of each sample according to step S13, and arrange p pieces of physical and chemical property data of each sample in a row order to obtain an input variable data matrix X. The number of samples is referred to as the sample capacity, which is denoted as n, that is, the input variable data matrix is a data matrix with n rows and p columns. The samples include waste biomass and their corresponding biochar.

Step S15: Encode identity multi-category labels of the waste biomass and the carbonized biochar thereof. Sources of the waste biomass include different categories such as straw, perishable garbage, pecan shells, and livestock manure, and the number of categories is denoted as w. Identity category information of the waste biomass is divided according to sources of waste biomass raw materials, and identity category information of the biochar depends on identity category information of its raw materials, so that identity categories yᵥ (v=1, 2, . . . , ω) of the waste biomass and the biochar prepared at different carbonization temperatures are labeled with numerical serial numbers 1, 2, 3, . . . , respectively. Identity category labels of the samples are labeled one by one according to row numbers of the input variable data matrix in step S14 to form an identity multi-category label column vector y of waste biomass and biochar samples.

Step S16: Construct high-dimensional multi-category biochar sample data, that is, combine the input variable data matrix X and the identity multi-category label column vector y to obtain high-dimensional multi-category biochar sample data {X,y}, where the input variable data matrix X and the identity category label column vector y have dimensions of n×p and n×1, respectively.

In this embodiment, in step S2, abnormality detection and standardization processing of input variables are performed: to ensure accuracy and reliability of modeling by the ensemble learning classifier, abnormality detection of sample data is performed. In addition, in order to reduce the influence of the dimension and order of magnitude of each physical and chemical property parameter on the determining model, the standardization processing of each input variable is performed, including the following steps.

Step S21: Perform abnormality detection, that is, implement an abnormal data identification algorithm based on a statistical method on random abnormal data that may exist in the input variable data matrix X, determine samples with abnormal data as outliers, and remove the outliers from the input variable data matrix X to obtain an input variable data matrix with the outliers removed.

Step S22: Standardize the input variable data matrix with the outliers removed to obtain a processed input variable data matrix, that is, calculate a mean and a standard deviation of each piece of physical and chemical property data based on the input variable data matrix with the outliers removed. The detailed process is as follows: Obtain the input variable data matrix X of the sample data from step S14, remove the outliers in step S21 and then denote as X^{⊗}, and calculate a mean x̅ₗ, and a standard deviation sₗ (l=1, 2, . . . , p) of the physical and chemical property data by column.

Standardization processing of input variables: based on the input variable data matrix X^{⊗} with the outliers removed, a column vector xₗ of each physical and chemical property parameter is standardized according to formula (1), and a standardized input variable data matrix is denoted as \tilde{X}. \tilde{x}ₗ is standardized physical and chemical property data.

$${\tilde{x}}_{l} = \frac{{x}_{l} - {\overline{x}}_{l}}{{s}_{l}} , l = 1 , 2 , … , p . \qquad ( 1 )$$

In this embodiment, in step S3, importance ranking and feature selection of input variables are performed: in order to reduce model complexity and improve model interpretability and prediction performance, input variables that have great influence on category attribute labels are identified and selected, and importance ranking and feature selection of the input variables are completed. A detailed process includes the following steps.

Step S31: Initialize weights and set parameters: initialize a weight of each input variable x₁, x₂, . . . , xₚ in the processed input variable data matrix to 0, that is, w⁽⁰⁾(xₗ)=0 (l=1, 2 . . . p), and set a number m of random sampling times, a number k₁ of nearest neighbor samples, and a variable importance threshold θ.

Step S32: Randomly select one sample from the processed input variable data matrix, and calculate a distance between the selected sample and the remaining samples; with the distance as a similarity measurement index, search out k₁ nearest neighbor samples with the same category attribute as the selected samples and forming a sample subset with the same category, and search out k₁ nearest neighbor samples with a category attribute different from that of the selected samples and forming a sample subset with different categories, specifically: obtain the processed input variable data matrix \tilde{X} from step S22, randomly select one sample \tilde{x}ᵢ therefrom, and calculate a distance d (l, \tilde{x}ᵢ, \tilde{x}ⱼ) between the sample and the remaining sample \tilde{x}ⱼ on an l^{th} (l=1, 2, . . . , p) input variable by formula (2). Taking the distance as a similarity measurement index, k₁ nearest neighbor (intra-class distance) samples with the same category attribute as \tilde{x}ᵢ are searched out to form a subset H_{i,j} (j=1, 2, . . . , k₁) and k₁ nearest neighbor (between-class distance) samples with ω−1 different category attributes are searched out to form a subset M_{i,j}(yᵥ) (j=1, 2, . . . , k₁, v=1, 2, . . . , ω, yᵥ≠yᵢ);

$$d ( l , {\tilde{x}}_{i} , {\tilde{x}}_{j} ) = \vert {x}_{i , l} - {x}_{j , l} \vert , l = 1 , 2 , … , p . \qquad ( 2 )$$

Step S33: Iteratively calculate the weight of each input variable in the processed input variable data matrix based on the sample subset with the same category and the sample subset with different categories. Specifically, the iterative weight w^{(h+1)}(xₗ) of the input variable xₗ is calculated by using formula (3), where l=1, 2, . . . , p, and h=0, 1, 2, . . . , m.

$${w}^{( h + 1 )} ( {x}_{l} ) = {w}^{( h )} ( {x}_{l} ) - \frac{{\sum }_{j = 1}^{{k}_{1}} d ( l , {\tilde{x}}_{i} , {H}_{i , j} )}{m \times {k}_{1}} + \frac{{\sum }_{{y}_{v} = 1 ( {y}_{v} \neq {y}_{i} )}^{{y}_{ω}} \frac{p ( {y}_{v} )}{1 - p ( {y}_{v} )} {\sum }_{j = 1}^{{k}_{1}} d ( l , {\tilde{x}}_{i} , {H}_{i , j} ( {y}_{v} ) )}{m \times {k}_{1}} . \qquad ( 3 )$$

p(yᵥ) is a prior probability of a sample subset with a category attribute being yᵥ in a training sample set.

Step S34: Determine a final weight of each input variable: repeat step S32 and step S33 until the number h+1 of random sampling times reaches m set in step S31, and obtain the final weight w^{(m)}(xₗ) (l=1, 2, . . . , p) of each input variable.

Step S35: Sort the input variables based on the final weight of each input variable in a descending order. Specifically, the final weight w^{(m)}(xₗ) (l=1, 2, . . . , p) of each input variable is obtained from step S34. The input variables are sequenced from high to low according to the final weights, and the sequenced input variables are denoted as xₗ′ (l=1, 2, . . . , p).

Step S36: Perform feature selection of input variables: compare the final weights of the sequenced input variables one by one with the variable importance threshold θ, and eliminate invalid and redundant input variables; calculate respective cumulative contribution rates ηᵣ by using formula (4) by increasing the number of input variables one by one from the remaining s input variables, and determine a number r of features selected by the input variables when ηᵣ is increased to 95% or more.

$${η}_{r} = \frac{{\sum }_{l = 1}^{r} {w}^{( m )} ( {x}_{l}^{′} )}{{\sum }_{l = 1}^{s} {w}^{( m )} ( {x}_{l}^{′} )} . \qquad ( 4 )$$

Step S37: Construct a feature data matrix based on the number of features selected by the input variables: obtain the processed input variable data matrix \tilde{X} from step S23, and construct a feature data matrix \tilde{X}′=[x₁′, x₂′, . . . , xᵣ′] of the input variables according to the sequenced input variable xₗ′ (l=1, 2, . . . , p) in step S35 and the number r of features determined in step S36.

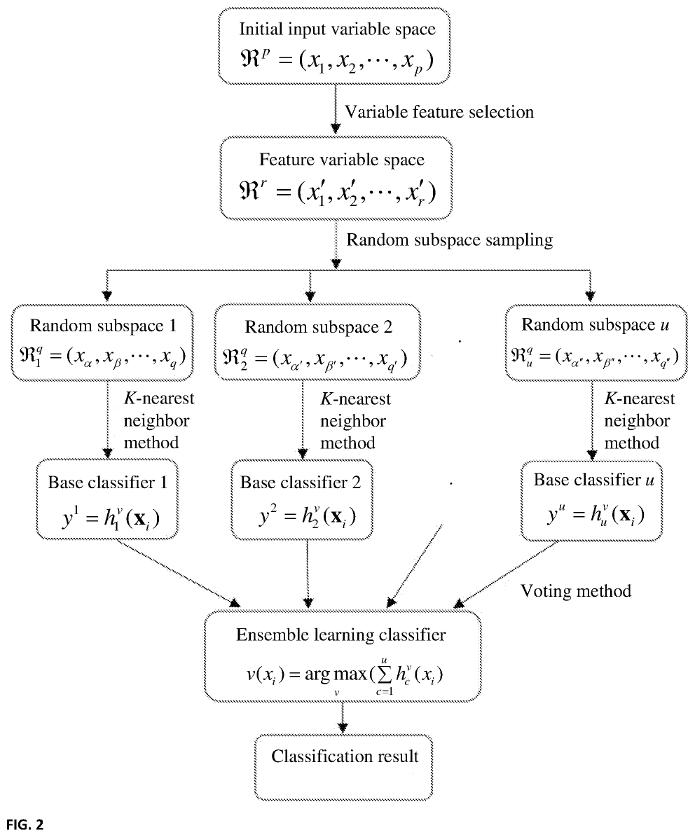

In this embodiment, in step S4, the random subspace nearest neighbor clustering ensemble learning classifier refers to a technology that combines a random subspace, K-nearest neighbor (KNN) clustering, ensemble learning and other methods and steps to improve prediction performance of the classifier. The random subspace can not only reduce the dimension of a feature space and the complexity of a base classifier, but also increase the diversity of each base classifier by different random permutations and combinations of input variables while a number of dimensions of the subspace is unchanged. In addition, KNN clustering can well deal with the similarity and relationship between sample individuals. Finally, different base classifiers are fused into an ensemble learning classifier with a stronger generalization ability by voting. However, noise of a sample data set, the number of dimensions of the subspace, the number of random sampling times and the number of KNN clustering nearest neighbors have significant influence on the performance of the ensemble learning classifier. Therefore, reasonable preprocessing and parameter adjustment need to be performed on the random subspace nearest neighbor clustering ensemble learning classifier. FIG. 2 is a functional structural diagram of a random subspace nearest neighbor clustering ensemble learning classifier. A detailed process is as follows.

In order to improve the processing adaptability of the ensemble learning classifier to diverse samples, a plurality of input variables with the same number of dimensions or different numbers of dimensions are first randomly selected from an input variable set after feature selection to form their own random subspaces, then a KNN clustering algorithm is used to construct a base classifier in each random subspace, and finally these base classifiers are fused into an ensemble learning classifier by using a voting method, thus improving the overall determining performance and robustness of the classifier.

Step S41: Create a random subspace: randomly select q features (1≤q<r) from an r-dimensional feature data matrix \tilde{X}′=[x₁′, x₂′, . . . , xᵣ′] of the input variables, and repeat this process for u times to obtain u q-dimensional subspace sample data matrices T_{c} (c=1, 2, . . . , u).

Step S42: Perform random subspace KNN clustering: obtain each q-dimensional random subspace sample matrix T_{c} (c=1, 2, . . . , u) from step S41, and implement the KNN clustering algorithm in the case of the number k₂ of nearest neighbor samples, to generate u base classifiers y^{(c)}=h_{c}^{v}(\tilde{x}ᵢ′, q, k₂)(\tilde{x}ᵢ′∈T_{c}).

Step S43: Perform ensemble learning of base classifiers: fuse the u base classifiers in step S42 by using a relative majority voting method, where the ensemble learning classifier thus constructed is shown in formula 5, that is, a result of ensemble learning is a category with the most votes, and if there are a plurality of categories with the highest votes at the same time, one category is randomly selected therefrom.

$$v = {argmax}_{v} ( {\sum }_{c = 1}^{u} {h}_{c}^{v} ( {\tilde{x}}_{i}^{′} , q , {k}_{2} ) ) . \qquad ( 5 )$$

Step S44: Optimize parameters of the random subspace nearest neighbor clustering ensemble learning classifier. The number q of dimensions of the random subspace in step S41 and k₂ in the KNN clustering algorithm in step S42 have great influence on the ensemble learning performance of the u base classifiers in step S43, and values of the above two parameters are positive integers. At present, with the determining accuracy of the ensemble learning classifier for sample data as an optimization index, grid-search is selected to find optimal q^{opt} and k₂^{opt}.

Step S45: Implement a random subspace nearest neighbor clustering ensemble learning classifier determining model: obtain the optimal q^{opt} and k₂^{opt} found in step S44, thus perform ensemble learning of the u base classifiers in step S43, and establish a biochar identity determination model shown in formula (6).

$${y}_{v} = {argmax}_{v} ( {\sum }_{c = 1}^{u} {h}_{c}^{v} ( {\tilde{x}}_{i}^{′} , {q}^{opt} , {k}_{2}^{opt} ) ) . \qquad ( 6 )$$

In this embodiment, step 200 specifically includes: performing abnormality detection and standardization processing on the physical and chemical property data of the samples to obtain processed physical and chemical property data of the samples; constructing a feature data matrix of the samples based on the processed physical and chemical property data and inputting the feature data matrix of the samples into a biochar identity determination model to obtain the identity information of the samples. A detailed process is as follows.

Step S201: Preprocess the samples: when a biochar identity category of the samples x_{new} needs to be determined, first perform outlier test of step S21 on the samples, obtain the mean x̅ₗ and the standard deviation s̅ₗ (l=1, 2, . . . , p) of each physical and chemical property parameter from step S22, substitute the mean and the standard deviation into formula (1) for standardization processing as \tilde{x}_{new}, and finally reduce the dimension to \tilde{x}_{new}′ according to r feature variables screened in step S36.

Step S202: Predict the biochar identity category of the samples by the base classifiers: obtain \tilde{x}_{new}′ from step S201, and substitute the same into the u base classifiers established in step S42 (the number q of dimensions of the random subspace of each base classifier and k₂ in the KNN clustering method come from q^{opt} and k₂^{opt} in step S45) to obtain a biochar identity category prediction result h_{c}^{v}(\tilde{x}_{new}′, q^{opt}, k₂^{opt}) (c=1, 2, . . . , u) of the samples in each base classifier.

Step S203: Determine the biochar identity of the sample: count u results of prediction of the biochar identity category of the sample in step S202, and use the relative majority voting method to substitute the results into the determining model of formula (6) in step S45 to determine the category with the most votes as the biochar identity category

$${y}_{v} = {argmax}_{v} ( {\sum }_{c = 1}^{u} {h}_{c}^{v} ( {\tilde{x}}_{new}^{′} , {q}^{opt} , {k}_{2}^{opt} ) )$$

of the sample.

The random subspace nearest neighbor clustering ensemble learning classifier for biochar identity determination according to the present disclosure is used to determine the biochar identity by using experimental data of physical and chemical properties of samples. The classifier can well complete a task of biochar identity determination, with prediction accuracy reaching 100%, and has the advantages of strong specificity, good repeatability, reliable results, and the like.

### Embodiment 2

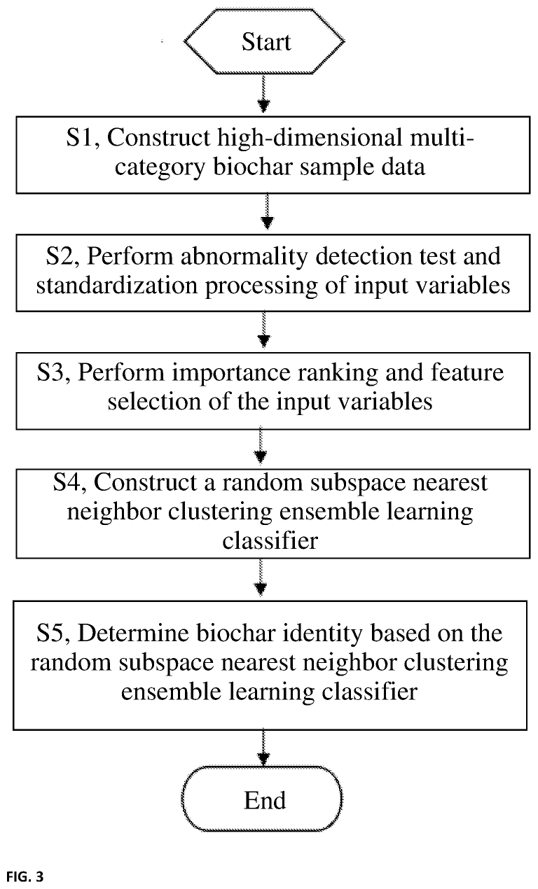

As shown in FIG. 3, a random subspace nearest neighbor clustering ensemble learning classifier for biochar identity determination according to the present disclosure includes the following steps sequentially performed.

Step S1: Construct high-dimensional multi-category biochar sample data: sort out data such as a plurality of important physical and chemical property parameters collected from experiments of waste biomass and the carbonized biochar thereof, and thus establish high-dimensional input vectors of the sample data and multi-category attribute labels of identity of the waste biomass and a carbide thereof.

In step S1, a method for constructing the high-dimensional multi-category biochar sample data includes the following steps.

Step S11: Screen physical and chemical property parameters of the waste biomass and the carbonized biochar thereof: select four types of physical and chemical property parameters, namely a carbon storage value, a fertility value, a pH scale and a particle size distribution based on application values of raw materials of the biochar and the carbonized biochar.

Step S12: Define specific physical and chemical property parameters. In this implementation, a hydrogen (H) content (%) and an organic carbon concentration Corg (%) are selected as carbon storage value indexes; a nitrogen (N) content, a phosphorus (P) content, a potassium (K) content (%), a sulfur(S) content (%), a calcium (Ca) content (%) and a magnesium (Mg) content (%) are selected as fertility value indexes; a pH scale is selected as an index of the degree of acidity and alkalinity; and a specific surface area BET (m²/g) and a pore volume Vpore (cm³/g) are selected as particle size distribution indexes.

Step S13: Arrange the high-dimensional input vectors: sequentially denote 11 physical and chemical property parameters defined in step S12 as x₁, x₂, . . . , x₁₁, and assemble in the form of row vectors to form input vectors x=[x₁, x₂, . . . , x₁₁] of the sample.

Step S14: Experimentally collect the input variable data matrix: select waste biomass from different regions and of different raw materials and biochar respectively prepared at three carbonization temperatures, namely a low temperature (350° C.), an intermediate temperate (500° C.) and a high temperate (650° C.), experimentally measure p=11 pieces of physical and chemical property parameter data defined in step S12, arrange an input vector x of each sample according to step S13, and arrange the input vectors of the samples in a row order to obtain an input variable data matrix X of waste biomass and biochar samples. In this implementation, the sample capacity is n=40.

Step S15: Encode identity multi-category labels of the waste biomass and the carbonized biochar thereof. Sources of the waste biomass include six categories, namely corn straw, rice straw, perishable garbage, pecan shells, cattle manure, and pig manure, that is, ω=6. Identity category information of the waste biomass is divided according to sources of waste biomass raw materials, and identity category information of the biochar depends on identity category information of its raw materials, so that identity categories yᵥ (v=1, 2, . . . , 6) of the waste biomass and the biochar prepared at three different carbonization temperatures are labeled with numerical serial numbers 1, 2, 3, 4, 5 and 6 respectively. Identity category labels of the samples are labeled one by one according to row numbers of the input variable data matrix X of biochar samples obtained in step S14 to form an identity multi-category label column vector y of biochar sample data.

Step S16: Construct high-dimensional multi-category biochar sample data: combine the input variable data matrix X and the identity category label column vector y to obtain sample data {X, y} of waste biomass and biochar. In this implementation, X and y have dimensions of 40×11 and 40×1, respectively.

Step S2: Perform abnormality detection and standardization processing of input variables: to ensure accuracy and reliability of data-driven modeling by the ensemble learning classifier, perform abnormality detection of sample data. In addition, in order to reduce the influence of the dimension and order of magnitude of each physical and chemical property parameter on the determining model, the standardization processing of each input variable is performed.

In step S2, a method for the abnormality detection and standardization processing of input variables includes the following steps.

Step S21: Perform abnormality detection: perform abnormal data identification based on a clustering density-based spatial clustering of applications with noise (DBSCAN) algorithm on random abnormal data that may exist in the input variable data matrix X, determine samples with abnormal data as outliers, and remove the outliers from the sample data. In this implementation, no outliers are found by implementing the DBSCAN algorithm.

Step S22: Calculate a mean and a standard deviation of each physical and chemical property parameter based on the input variable data matrix with the outliers removed: obtain the input variable matrix X of the sample data from step S14, where since no outlier is detected in step S21, after the outliers are removed, X^{⊗}=X; and then calculate the mean x̅ₗ and the standard deviation sₗ (l=1, 2, . . . , 11) of each physical and chemical property parameter by column.

Step S23: Standardize input variables: based on the input variable data matrix X, standardize a column vector xₗ of each physical and chemical property parameter according to formula (1), and denote a standardized input variable data matrix as \tilde{X}.

Step S3: Perform importance ranking and feature selection of input variables: in order to reduce model complexity and improve model interpretability and prediction performance, identify and select input variables that have great influence on category attribute labels, and complete importance ranking and feature selection of the input variables.

In step S3, a method for the importance ranking and feature selection of input variables includes the following steps.

Step S31: Initialize weights and set parameters: initialize a weight of each input variable x₁, x₂, . . . , x₁₁ to 0, that is, w⁽⁰⁾(xₗ)=0 (l=1, 2, . . . , 11), and set a number of random sampling times to m=n=40, a number of nearest neighbor samples to k₁=3, and a variable importance threshold to 0.

Step S32: Set up a sample subset H_{i,j}(yᵢ) with the same category and a sample subset M_{i,j}(yᵥ) with different categories: obtain the standardized input variable data matrix \tilde{X} from step S22, randomly select one sample \tilde{x}ᵢ therefrom, and calculate a distance d(l, \tilde{x}ᵢ, \tilde{x}ⱼ) between the sample and the remaining sample \tilde{x}ⱼ on an l^{th} (l=1, 2, . . . , 11) input variable by formula (2). Taking the distance as a similarity measurement index, k₁ nearest neighbor (intra-class distance) samples with the same category attribute as \tilde{x}ᵢ are searched out to form a subset H_{i,j} (j=1, 2, 3) and k₁=3 nearest neighbor (between-class distance) samples with ω−1=5 different category attributes are searched out to form a subset M_{i,j}(yᵥ) (j=1, 2, 3, v=1, 2, . . . , 6, yᵥ≠yᵢ).

Step S33: Calculate an iterative weight of each input variable: calculate the iterative weight w^{(h+1)}(xₗ) of the input variable xₗ by using formula (3), where l=1, 2, . . . , 11, and h=0, 1, 2, L, 39. In this implementation, prior probabilities p(yᵥ) of the six categories are 0.2, 0.2, 0.1, 0.1, 0.2 and 0.2, respectively.

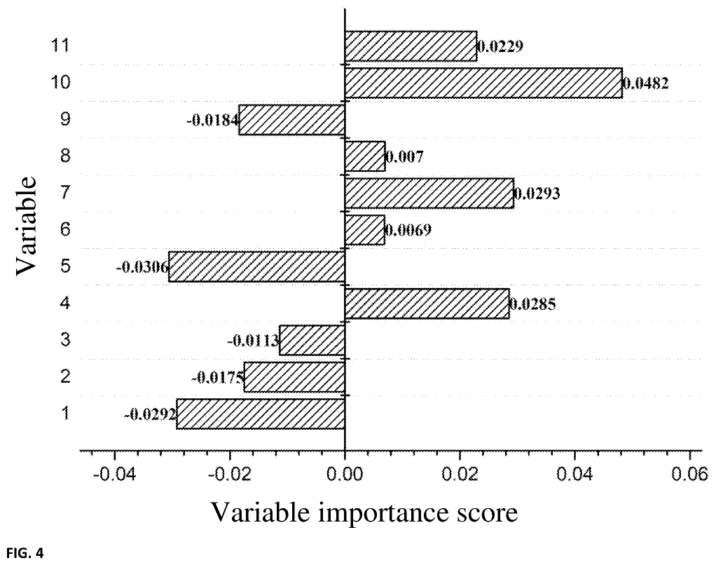

Step S34: Determine a final weight of each input variable: repeat step S32 and step S33 until the number h+1 of random sampling times reaches m=40 set in step S31, and obtain the final weight w⁽⁴⁰⁾(xₗ) (l=1, 2, . . . , 11) of each input variable. In this implementation, the final weight result of each input variable is shown in FIG. 4.

Step S35: Perform importance ranking of input variables: obtain the final weight w⁽⁴⁰⁾(xₗ) (l=1, 2, . . . , 11) of each input variable from step S34, where the input variable with a greater weight is more important. The input variables are sequenced from high to low according to the weights, and the sequenced input variables are denoted as x₁′=x₁₀, x₂′=x₇, x₃′=x₄, x₄′=x₁₁, x₅′=x₈, x₆′=x₆, x₇′=x₃, x₈′=x₂, x₉′=x₉, x₁₀′=x₁, and x₁₁′=x₅.

Step S36: Perform feature selection of input variables: compare the final weights of the input variables one by one with the variable importance threshold θ=0, and eliminate five invalid and redundant input variables: x₁₀′=x₁, x₈′=x₂, x₇′=x₃, x₁₁′=x₅, and x₉′=x₉; and calculate respective cumulative contribution rates η, by using formula (4) by increasing the number of input variables one by one from the remaining s=11−5=6 input variables. In this implementation, a value ηᵣ=96% is taken to determine the number r=6 of features of the input variables.

Step S37: Construct a feature data matrix of the input variables: obtain the standardized input variable data matrix \tilde{X} from step S23, and construct a feature data matrix \tilde{X}′=[x₁′, x₂′, . . . , x₆′] of the input variables according to the sequenced input variable xₗ′ (l=1, 2, . . . , 11) in step S35 and the number r=6 of features determined in step S36.

Step S4: Construct a random subspace nearest neighbor clustering ensemble learning classifier: in order to improve the processing adaptability of the ensemble learning classifier to diverse samples, first randomly select, from an input variable set after feature selection, a plurality of input variables with the same number of dimensions or different numbers of dimensions to form their own random subspaces, then use a KNN clustering algorithm to construct a base classifier in each random subspace, and finally fuse these base classifiers into an ensemble learning classifier by using a voting method, thus improving the overall determining performance and robustness of the classifier.

In step S4, a method for constructing the random subspace nearest neighbor clustering ensemble learning classifier includes the following steps.

Step S41: Create a random subspace: randomly select q=3 features from an r=6−dimensional feature data matrix \tilde{X}′=[x₁′, x₂′, . . . , x₆′] of the input variables, and repeat this process for u=30 times to obtain 30 three-dimensional subspace sample data matrices T_{c} (c=1, 2, . . . , 30).

Step S42: Perform random subspace KNN clustering: obtain each q=3-dimensional random subspace sample matrix T_{c} (c=1, 2, . . . , 30) from step S41, and implement the KNN clustering algorithm in the case of the number k₂=3 of nearest neighbor samples to generate 30 base classifiers y^{(c)}=h_{c}^{v}(\tilde{x}ᵢ′, q, k₂)(\tilde{x}ᵢ′∈T_{c}, c=1, 2, . . . , 30).

Step S43: Perform ensemble learning of base classifiers: fuse the u=30 base classifiers in step S42 by using a relative majority voting method, where an output result

$$v = {argmax}_{v} ( {\sum }_{c = 1}^{30} {h}_{c}^{v} ( {\tilde{x}}_{i}^{′} , q , {k}_{2} ) )$$

of the ensemble learning classifier thus constructed is the category with the most votes, as shown in formula (5). If there are a plurality of categories with the highest votes at the same time, one category is randomly selected therefrom.

Step S44: Optimize parameters of the random subspace nearest neighbor clustering ensemble learning classifier. The number q of dimensions of the random subspace in step S41 and k₂ in the KNN clustering algorithm in step S42 have great influence on the ensemble learning performance of the u base classifiers in step S43, and values of the above two parameters are positive integers. At present, with the determining accuracy

$$φ = {\sum }_{v = 1}^{6} {n}_{v} / n$$

(nᵥ is the number of samples of the V^{th} category correctly determined) of the ensemble learning classifier for sample data as an optimization index, grid-search is selected to find optimal q^{opt} and k₂^{opt}. In this implementation, set q has an optimization range of [1,5] with a stride of 1, and k₂ has an optimization range of [1,6] with a stride of 1. Different level values of q and k₂ form a total of 30 permutations and combinations, there are a plurality of groups of optimization results that meet requirements for maximum φ, and q^{opt}=3 and k₂^{opt}=3 are obtained.

Step S45: Implement a random subspace nearest neighbor clustering ensemble learning classifier determining model: obtain the optimal q^{opt}=3 and k₂^{opt}=3 found in step S44, thus perform ensemble learning of the u=30 base classifiers in step S43, and establish a biochar identity determination model

$${y}_{v} = {argmax}_{v} ( {\sum }_{c = 1}^{30} {h}_{c}^{v} ( {\tilde{x}}_{i}^{′} , {q}^{opt} , {k}_{2}^{opt} ) )$$

shown in formula (6).

Step S5: Determine biochar identity based on the random subspace nearest neighbor clustering ensemble learning classifier: determine biochar identity of any sample individual by using the random subspace nearest neighbor clustering ensemble learning classifier constructed in step S4.

In step S5, a method for determining the biochar identity based on the random subspace nearest neighbor clustering ensemble learning classifier includes the following steps.

Step S51: Preprocess a sample: when a biochar identity category of a new sample x_{new} needs to be determined, first perform outlier test of step S21 on the sample, obtain a mean x̅ₗ and a standard deviation sₗ (l=1, 2, . . . , 11) of each physical and chemical property parameter from step S22, substitute the mean and the standard deviation into formula (1) for standardization processing as \tilde{x}_{new}, and finally reduce the dimension to \tilde{x}_{new}′ according to r=6 feature variables screened in step S36.

Step S52: Predict the biochar identity category of the sample by the base classifiers: obtain \tilde{x}_{new}′ from step S51, and substitute the same into the u=30 base classifiers established new in step S42 (the number q of dimensions of the random subspace of each base classifier and k₂ in the KNN clustering method come from q^{opt}=3 and k₂^{opt}=3 in step S45) to obtain a biochar identity category prediction result h_{c}^{v}(\tilde{x}_{new}′, q^{opt}, k₂^{opt}) (c=1, 2, . . . , 30) of the sample in each base classifier.

Step S53: Determine biochar identity of the sample: count u=30 results of prediction of the biochar identity category of the sample in step S52, and use the relative majority voting method to substitute the results into the determining model of formula (6) in step S45 to determine the category with the most votes as the biochar identity category

$${y}_{v} = {argmax}_{v} ( {\sum }_{c = 1}^{u} {h}_{c}^{v} ( {\tilde{x}}_{new}^{′} , {q}^{opt} , {k}_{2}^{opt} ) )$$

of the sample.

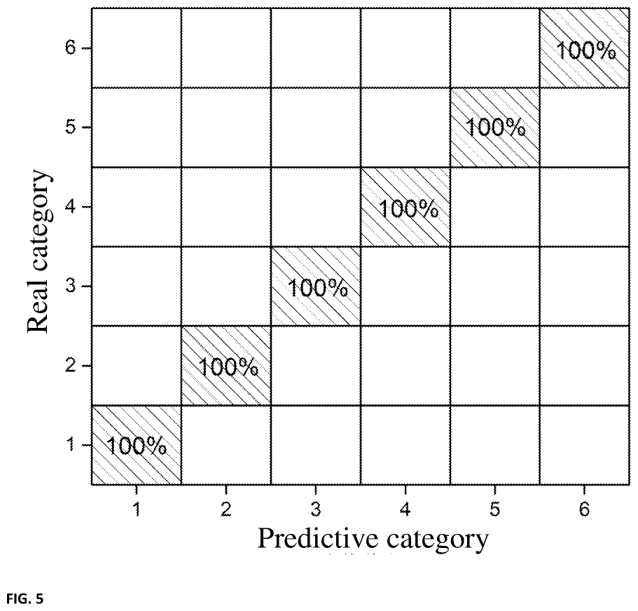

The determining result of biochar identity in this implementation is shown in FIG. 5, with the determining accuracy for each category being φᵥ=100% (v=1, 2, . . . , 6) and the determining accuracy of overall sample data being φ=100%.

### Embodiment 3

To implement the method corresponding to Embodiment 1 and achieve corresponding functions and technical effects, a system for determining biochar identity is provided below.

The system for determining biochar identity according to this embodiment includes:
a sample data acquisition module, configured to obtain physical and chemical property data of the samples to be identified, where the samples include waste biomass and their corresponding biochar. The biochar is a solid material obtained by carbonizing the waste biomass, and the physical and chemical property data includes a hydrogen content, an organic carbon concentration, a nitrogen content, a phosphorus content, a potassium content, a pH scale, a specific surface area, and a pore volume; and
a biochar identity information determining module, configured to input the physical and chemical property data of the samples into the biochar identity determination model to obtain identity information of the samples.

A process for determining the biochar identity determination model includes the following steps:
constructing high-dimensional multi-category biochar sample data, where the high-dimensional multi-category biochar sample data includes an input variable data matrix and an identity multi-category label column vector;
performing abnormality detection and standardization processing on the input variable data matrix in the high-dimensional multi-category biochar sample data to obtain a processed input variable data matrix;
constructing a feature data matrix based on the processed input variable data matrix; and
obtaining a random subspace nearest neighbor clustering ensemble learning classifier based on the feature data matrix, the sample identity multi-category label column vector and a random subspace nearest neighbor clustering ensemble learning algorithm, where the random subspace nearest neighbor clustering ensemble learning classifier is the biochar identity determination model.

### Embodiment 4

An embodiment of the present disclosure provides an electronic device, including a memory and a processor, where the memory is configured to store a computer program, and the processor runs the computer program to enable the electronic device to perform the method for determining biochar identity according to Embodiment 1. Optionally, the electronic device described above may be a server.

Embodiments of the description are described in a progressive manner, each embodiment focuses on the difference from other embodiments, and for the same and similar parts between the embodiments, reference may be made to each other. Since the system disclosed in an embodiment corresponds to the method disclosed in an embodiment, the description is relatively simple, and for related contents, references can be made to the description of the method.

Particular examples are used herein for illustration of principles and implementations of the present disclosure. The descriptions of the above embodiments are merely used for assisting in understanding the method of the present disclosure and its core ideas. In addition, those of ordinary skill in the art can make modifications in terms of specific implementations and the scope of application in accordance with the ideas of the present disclosure. In conclusion, the content of the description shall not be construed as limitations to the present disclosure.

What is claimed is:

1. A method for determining biochar identity, comprising:
obtaining physical and chemical property data of a sample to be identified, wherein the sample comprises waste biomass and biochar corresponding to the waste biomass, the biochar is a solid material obtained by carbonizing the waste biomass, and the physical and chemical property data comprises a hydrogen content, an organic carbon concentration, a nitrogen content, a phosphorus content, a potassium content, a pH scale, a specific surface area, and a pore volume; and
inputting the physical and chemical property data of the sample into a biochar identity determination model to obtain identity information of the sample, wherein
a process for determining the biochar identity determination model comprises the following steps:
constructing high-dimensional multi-category biochar sample data, wherein the high-dimensional multi-category biochar sample data comprises an input variable data matrix and an identity multi-category label column vector;
performing abnormality detection and standardization processing on the input variable data matrix in the high-dimensional multi-category biochar sample data to obtain a processed input variable data matrix;
constructing a feature data matrix based on the processed input variable data matrix; and
obtaining a random subspace nearest neighbor clustering ensemble learning classifier based on the feature data matrix, the sample identity multi-category label column vector and a random subspace nearest neighbor clustering ensemble learning algorithm, wherein the random subspace nearest neighbor clustering ensemble learning classifier is the biochar identity determination model.

2. The method for determining biochar identity according to claim 1, wherein the constructing high-dimensional multi-category biochar sample data specifically comprises:
screening physical and chemical property parameters of the sample, wherein the sample comprises waste biomass and biochar obtained by carbonizing the waste biomass, and the physical and chemical property parameters comprise a carbon storage value, a fertility value, a pH scale, and particle size distribution;
defining specific physical and chemical property parameters to obtain a plurality of physical and chemical property indexes, wherein the plurality of physical and chemical property indexes comprise a hydrogen content, an organic carbon concentration, a nitrogen content, a phosphorus content, a potassium content, a pH scale, a specific surface area, and a pore volume;
sequentially numbering the physical and chemical property indexes, and assembling in the form of row vectors to form input vectors of the sample;
selecting waste biomass from different regions and biochar respectively prepared at different carbonization temperatures, experimentally measuring a value corresponding to each physical and chemical property index, and arranging physical and chemical property data corresponding to each sample in a row order based on the input vector form of the sample to obtain the input variable data matrix, wherein the input variable data matrix is a data matrix with n rows and p columns, p represents a number of physical and chemical property indexes, n represents a number of samples, and the samples comprise waste biomass and biochar corresponding to the waste biomass;
labeling identity category labels of the samples one by one according to row numbers of the input variable data matrix to form the sample identity multi-category label column vector; and
constructing the high-dimensional multi-category biochar sample data based on the input variable data matrix and the identity multi-category label column vector.

3. The method for determining biochar identity according to claim 2, wherein the performing abnormality detection and standardization processing on the input variable data matrix in the high-dimensional multi-category biochar sample data to obtain a processed input variable data matrix specifically comprises:
determining, based on an abnormal data identification algorithm in a statistical method, samples with abnormal data in the input variable data matrix as outliers and removing the outliers to obtain an input variable data matrix with the outliers removed; and
standardizing the input variable data matrix with the outliers removed, to obtain the processed input variable data matrix.

4. The method for determining biochar identity according to claim 3, wherein the constructing a feature data matrix based on the processed input variable data matrix specifically comprises:
step 1: initializing a weight of each input variable in the processed input variable data matrix to 0, and setting a number m of random sampling times, a number k₁ of nearest neighbor samples, and a variable importance threshold θ;
step 2: randomly selecting one sample from the processed input variable data matrix, and calculating a distance between the selected sample individual and the remaining sample individuals; with the distance as a similarity measurement index, searching out k₁ nearest neighbor sample individuals with the same category attribute as the selected sample individual and forming a sample subset with the same category, and searching out k₁ nearest neighbor sample individuals with a category attribute different from that of the selected sample individual and forming a sample subset with different categories;
step 3: iteratively calculating the weight of each input variable in the processed input variable data matrix based on the sample subset with the same category and the sample subset with different categories;
step 4: repeating step 2 and step 3 until the number of random sampling times reaches the set number m of random sampling times, and obtaining a final weight of each input variable in the processed input variable data matrix;
step 5: sorting the input variables based on the final weight of each input variable in a descending order;
step 6: comparing the final weights of the sequenced input variables one by one with the variable importance threshold θ, eliminating invalid and redundant input variables to obtain the remaining sequenced input variables, calculating a cumulative contribution rate by increasing the number of input variables one by one from the remaining sequenced input variables, and determining a number of features selected by the input variables when the cumulative contribution rate is greater than a set value; and
step 7: constructing the feature data matrix based on the number of features selected by the input variables.

5. The method for determining biochar identity according to claim 4, wherein the obtaining a random subspace nearest neighbor clustering ensemble learning classifier based on the feature data matrix, the sample identity multi-category label column vector and a random subspace nearest neighbor clustering ensemble learning algorithm, wherein the random subspace nearest neighbor clustering ensemble learning classifier is the biochar identity determination model specifically comprises:
randomly selecting q features from the feature data matrix and repeating for u times to obtain u q-dimensional subspace sample data matrices;
performing a K-nearest neighbor (KNN) clustering algorithm on each q-dimensional subspace sample data matrix in the case of the number k₂ of nearest neighbor samples, to generate u base classifiers;
fusing the u base classifiers by using a relative majority voting method to obtain an ensemble learning classifier; and
performing grid-search to find optimal q and k₂ with determining accuracy of the ensemble learning classifier on sample data as an optimization index, and then obtaining the random subspace nearest neighbor clustering ensemble learning classifier, wherein the sample data comprises the feature data matrix and the sample identity multi-category label column vector.

6. The method for determining biochar identity according to claim 1, wherein the inputting the physical and chemical property data of the sample into a biochar identity determination model to obtain identity information of the sample specifically comprises:
performing abnormality detection and standardization processing on the physical and chemical property data of the sample to obtain processed physical and chemical property data of the sample;
constructing a feature data matrix of the sample based on the processed physical and chemical property data of the sample; and
inputting the feature data matrix of the sample into the biochar identity determination model to obtain the identity information of the sample.

7. A system for determining biochar identity, comprising:
a sample data acquisition module, configured to obtain physical and chemical property data of a sample to be identified, wherein the sample comprises waste biomass and biochar corresponding to the waste biomass, the biochar is a solid material obtained by carbonizing the waste biomass, and the physical and chemical property data comprises a hydrogen content, an organic carbon concentration, a nitrogen content, a phosphorus content, a potassium content, a pH scale, a specific surface area, and a pore volume; and
a biochar identity information determining module, configured to input the physical and chemical property data of the sample into the biochar identity determination model to obtain identity information of the sample, wherein
a process for determining the biochar identity determination model comprises the following steps:
constructing high-dimensional multi-category biochar sample data, wherein the high-dimensional multi-category biochar sample data comprises an input variable data matrix and an identity multi-category label column vector;
performing abnormality detection and standardization processing on the input variable data matrix in the high-dimensional multi-category biochar sample data to obtain a processed input variable data matrix;
constructing a feature data matrix based on the processed input variable data matrix; and
obtaining a random subspace nearest neighbor clustering ensemble learning classifier based on the feature data matrix, the sample identity multi-category label column vector and a random subspace nearest neighbor clustering ensemble learning algorithm, wherein the random subspace nearest neighbor clustering ensemble learning classifier is the biochar identity determination model.

8. An electronic device, comprising a memory and a processor, wherein the memory is configured to store a computer program, and the processor runs the computer program to enable the electronic device to perform the method for determining biochar identity according to claim 1.

9. The electronic device according to claim 8, wherein the constructing high-dimensional multi-category biochar sample data specifically comprises:
screening physical and chemical property parameters of the sample, wherein the sample comprises waste biomass and biochar obtained by carbonizing the waste biomass, and the physical and chemical property parameters comprise a carbon storage value, a fertility value, a pH scale, and particle size distribution;
defining specific physical and chemical property parameters to obtain a plurality of physical and chemical property indexes, wherein the plurality of physical and chemical property indexes comprise a hydrogen content, an organic carbon concentration, a nitrogen content, a phosphorus content, a potassium content, a pH scale, a specific surface area, and a pore volume;
sequentially numbering the physical and chemical property indexes, and assembling in the form of row vectors to form input vectors of the sample;
selecting waste biomass from different regions and biochar respectively prepared at different carbonization temperatures, experimentally measuring a value corresponding to each physical and chemical property index, and arranging physical and chemical property data corresponding to each sample in a row order based on the input vector form of the sample to obtain the input variable data matrix, wherein the input variable data matrix is a data matrix with n rows and p columns, p represents a number of physical and chemical property indexes, n represents a number of samples, and the samples comprise waste biomass and biochar corresponding to the waste biomass;
labeling identity category labels of the samples one by one according to row numbers of the input variable data matrix to form the sample identity multi-category label column vector; and
constructing the high-dimensional multi-category biochar sample data based on the input variable data matrix and the identity multi-category label column vector.

10. The electronic device according to claim 9, wherein the performing abnormality detection and standardization processing on the input variable data matrix in the high-dimensional multi-category biochar sample data to obtain a processed input variable data matrix specifically comprises:
determining, based on an abnormal data identification algorithm in a statistical method, samples with abnormal data in the input variable data matrix as outliers and removing the outliers to obtain an input variable data matrix with the outliers removed; and
standardizing the input variable data matrix with the outliers removed, to obtain the processed input variable data matrix.

11. The electronic device according to claim 10, wherein the constructing a feature data matrix based on the processed input variable data matrix specifically comprises:
step 1: initializing a weight of each input variable in the processed input variable data matrix to 0, and setting a number m of random sampling times, a number k₁ of nearest neighbor samples, and a variable importance threshold θ;
step 2: randomly selecting one sample from the processed input variable data matrix, and calculating a distance between the selected sample individual and the remaining sample individuals; with the distance as a similarity measurement index, searching out k₁ nearest neighbor sample individuals with the same category attribute as the selected sample individual and forming a sample subset with the same category, and searching out k₁ nearest neighbor sample individuals with a category attribute different from that of the selected sample individual and forming a sample subset with different categories;
step 3: iteratively calculating the weight of each input variable in the processed input variable data matrix based on the sample subset with the same category and the sample subset with different categories;
step 4: repeating step 2 and step 3 until the number of random sampling times reaches the set number m of random sampling times, and obtaining a final weight of each input variable in the processed input variable data matrix;
step 5: sorting the input variables based on the final weight of each input variable in a descending order;
step 6: comparing the final weights of the sequenced input variables one by one with the variable importance threshold θ, eliminating invalid and redundant input variables to obtain the remaining sequenced input variables, calculating a cumulative contribution rate by increasing the number of input variables one by one from the remaining sequenced input variables, and determining a number of features selected by the input variables when the cumulative contribution rate is greater than a set value; and
step 7: constructing the feature data matrix based on the number of features selected by the input variables.

12. The electronic device according to claim 11, wherein the obtaining a random subspace nearest neighbor clustering ensemble learning classifier based on the feature data matrix, the sample identity multi-category label column vector and a random subspace nearest neighbor clustering ensemble learning algorithm, wherein the random subspace nearest neighbor clustering ensemble learning classifier is the biochar identity determination model specifically comprises:
randomly selecting q features from the feature data matrix and repeating for u times to obtain u q-dimensional subspace sample data matrices;
performing a K-nearest neighbor (KNN) clustering algorithm on each q-dimensional subspace sample data matrix in the case of the number k₂ of nearest neighbor samples, to generate u base classifiers;
fusing the u base classifiers by using a relative majority voting method to obtain an ensemble learning classifier; and
performing grid-search to find optimal q and k₂ with determining accuracy of the ensemble learning classifier on sample data as an optimization index, and then obtaining the random subspace nearest neighbor clustering ensemble learning classifier, wherein the sample data comprises the feature data matrix and the sample identity multi-category label column vector.

13. The electronic device according to claim 8, wherein the inputting the physical and chemical property data of the sample into a biochar identity determination model to obtain identity information of the sample specifically comprises:
performing abnormality detection and standardization processing on the physical and chemical property data of the sample to obtain processed physical and chemical property data of the sample;
constructing a feature data matrix of the sample based on the processed physical and chemical property data of the sample; and
inputting the feature data matrix of the sample into the biochar identity determination model to obtain the identity information of the sample.
